SEQUENCE LISTING (14 sequences)

SEQ ID NO: 1 - DNA, 20 bp, synthetic construct
  source 1..20: mol_type = other DNA
gacaggcatctcacgaaccg

SEQ ID NO: 2 - DNA, 20 bp, synthetic construct
  source 1..20: mol_type = other DNA
ccaacgggtctgctggttta

SEQ ID NO: 3 - DNA, 20 bp, synthetic construct
  source 1..20: mol_type = other DNA
acgagaccaagaactgcccc

SEQ ID NO: 4 - DNA, 20 bp, synthetic construct
  source 1..20: mol_type = other DNA
agtgtctcccttgggtccct

SEQ ID NO: 5 - DNA, 20 bp, synthetic construct
  source 1..20: mol_type = other DNA
cccacaaaccccaaaagcgt

SEQ ID NO: 6 - DNA, 21 bp, synthetic construct
  source 1..21: mol_type = other DNA
gggtagaagggatttgtgcgc

SEQ ID NO: 7 - DNA, 20 bp, synthetic construct
  source 1..20: mol_type = other DNA
cggttccaggggttgtgtca

SEQ ID NO: 8 - DNA, 20 bp, synthetic construct
  source 1..20: mol_type = other DNA
gacaccaaagccgggaaagc

SEQ ID NO: 9 - DNA, 21 bp, synthetic construct
  source 1..21: mol_type = other DNA
cactgcgtggcgaatgtaaac

SEQ ID NO: 10 - DNA, 21 bp, synthetic construct
  source 1..21: mol_type = other DNA
cgttgagcaagtttacggggt

SEQ ID NO: 11 - DNA, 20 bp, synthetic construct
  source 1..20: mol_type = other DNA
gaatgccagcctgattgccc

SEQ ID NO: 12 - DNA, 20 bp, synthetic construct
  source 1..20: mol_type = other DNA
tgtcccagttgaaggcccag

SEQ ID NO: 13 - DNA, 20 bp, synthetic construct
  source 1..20: mol_type = other DNA
gaatgccagcctgattgccc

SEQ ID NO: 14 - DNA, 20 bp, synthetic construct
  source 1..20: mol_type = other DNA
tgtcccagttgaaggcccag

(19) United States
(12) Patent Application Publication
(10) Pub. No.: US 2024/0180805 A1
(43) Pub. Date: Jun. 6, 2024

(54) COMPOSITION FOR INHIBITING WRINKLE

(71) Applicants: YUKI GOSEI KOGYO CO., LTD., Tokyo (JP); KYUSHU UNIVERSITY, NATIONAL UNIVERSITY CORPORATION, Fukuoka-shi (JP)

(72) Inventors: Tetsuji NODA, Tokyo (JP); Yoshinori KATAKURA, Fukuoka-shi (JP)

(73) Assignees: YUKI GOSEI KOGYO CO., LTD., Tokyo (JP); KYUSHU UNIVERSITY, NATIONAL UNIVERSITY CORPORATION, Fukuoka-shi (JP)

(21) Appl. No.: 18/456,798

(22) Filed: Aug. 28, 2023

(30) Foreign Application Priority Data
Aug. 30, 2022 (JP) 2022137311

(51) Int. Cl. A61K 8/44; A61K 8/98; A61Q 19/08

(52) U.S. Cl. CPC A61K 8/44; A61K 8/98; A61Q 19/08

(57) ABSTRACT

[OBJECT]

The object of the present invention is to provide an ingredient for inhibiting wrinkle with excellent safety.

[MEANS FOR SOLUTION]

The problem can be solved by a composition for inhibiting wrinkle, comprising β-alanine or a salt thereof as an active ingredient, of the present invention.

### TECHNICAL FIELD

The present invention relates to a composition for inhibiting wrinkle.

### BACKGROUND ART

The skin forms the outermost layer of the human body and plays an important role in protecting the body. On the other hand, the skin is also important in beauty care because its appearance is visually perceived. That is to say, there is a growing interest in the prevention of skin aging in terms of health and beauty.

Glucosylceramide (Patent literature 1), fullerenes (Patent literature 2), extracts of Pogonatherum crinitum (Patent literature 3), and the like have been reported as ingredients that improve symptoms such as wrinkles and sagging of the skin due to aging.

### CITATION LIST

### Patent Literature

[Patent literature 1] JP 2013-241370 A
[Patent literature 2] WO 2009/113426
[Patent literature 3] JP 2011-190212 A

### SUMMARY OF INVENTION

### Technical Problem

However, it is expected to develop an ingredient for inhibiting wrinkle with excellent safety. Therefore, the object of the present invention is to provide an ingredient for inhibiting wrinkle with excellent safety.

### Solution to Problem

The present inventors have conducted intensive studies into an ingredient for inhibiting wrinkle with excellent safety, as a result, surprisingly found that β-alanine exhibits an excellent wrinkle-inhibiting effect.

The present invention is based on the above findings.

Accordingly, the present invention relates to:
[1] a composition for inhibiting wrinkle, comprising β-alanine or a salt thereof as an active ingredient
[2] the composition for inhibiting wrinkle of the item [1], wherein an inhibition of wrinkle is due to, in dermal cells, a promotion of type I collagen component (COL1A1), inhibition of type I collagenase, promotion of elastin synthesis, promotion of microfibrillar associated protein (MFAP4), inhibition of elastase, promotion of hyaluronic acid synthase, or inhibition of hyaluronidase,
[3] a composition for inhibiting wrinkle, comprising a culture product secreted from muscle cells by β-alanine or salt thereof,
[4] the composition for inhibiting wrinkle of the item [3], wherein an inhibition of wrinkle is due to, in dermal cells, a promotion of type I collagen component (COL1A1), inhibition of type I collagenase, promotion of elastin synthesis, promotion of microfibrillar associated protein (MFAP4), inhibition of elastase, promotion of hyaluronic acid synthase, or inhibition of hyaluronidase,
[5] the composition for inhibiting wrinkle of the item [3], wherein the culture product is an exosome, and
[6] the composition for inhibiting wrinkle of any one of the items [1] to [5], wherein the composition is a food composition or a cosmetic composition.

Further, the present specification discloses:
[7] a method for inhibiting wrinkle, comprising a step of administrating an effective amount of β-alanine or a salt thereof to a subject (the method may be conducted as a medical practice, or a medical practice may be excluded from the method),
[8] a method for inhibiting wrinkle, comprising a step of administrating an effective amount of a culture product secreted from muscle cells by β-alanine or salt thereof to a subject (the method may be conducted as a medical practice, or a medical practice may be excluded from the method),
[9] A method for inhibiting wrinkle of the item [8], wherein the culture product is an exosome (the method may be conducted as a medical practice, or a medical practice may be excluded from the method),
[10] a culture product produced by bringing β-alanine or salt thereof into contact with muscle cells, and
[11] an exosome produced by bringing β-alanine or salt thereof into contact with muscle cells.

### Advantageous Effects of Invention

According to the composition for inhibiting wrinkle of the present invention, the wrinkle of skin can be effectively inhibited. The composition for inhibiting wrinkle can be used as a food composition or a cosmetic composition. The composition for inhibiting wrinkle with excellent safety can be provided by the present invention.

### BRIEF DESCRIPTION OF THE DRAWINGS

FIG. 1 is a graph showing that the effect of β-alanine on hyaluronic acid synthase (HAS2) in dermal fibroblasts was examined.

FIG. 2 is graphs showing that the effects via the supernatant of C2C12 cells of β-alanine on COL1A1 and MMP-1 in dermal fibroblasts was examined.

FIG. 3 is graphs showing that the effects via the supernatant of C2C12 cells of β-alanine on ELN and ELANE in dermal fibroblasts was examined.

FIG. 4 is graphs showing that the effects via the supernatant of C2C12 cells of β-alanine on HAS2 and HYAL1 in dermal fibroblasts was examined.

FIG. 5 is graphs showing the effects on COL1A1, HAS2, ELN, and MFAP4 when exosomes secreted from C2C12 cells by β-alanine were added to dermal fibroblasts.

FIG. 6 is graphs showing the effects on MMP-1, HYAL1, and ELANE when exosomes secreted from C2C12 cells by β-alanine were added to sdermal fibroblasts.

FIG. 7 is a graph showing the measurement of the area of COL1A1 protein positivity from photographs of immunostaining for COL1A1 protein in dermal fibroblasts treated with exosomes.

FIG. 8 is photographs of immunostaining of COL1A1 protein in dermal fibroblasts treated with exosomes.

### DESCRIPTION OF EMBODIMENTS

The composition for inhibiting wrinkle comprises β-alanine or a salt thereof as an active ingredient.

β-alanine is represented by the following formula [1]:

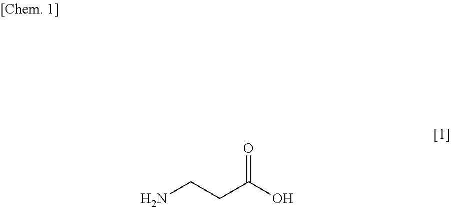

and is also called 3-aminopropanoic acid. Extracts, concentrates, or purified products from foods or natural products that contain relatively large amounts of β-alanine, can be used, as β-alanine comprised in the composition for inhibiting wrinkle of the present invention. In addition, synthetic β-alanine may be used. For example, β-alanine can be synthesized from β-propiolactone by the β-alanine synthesis method (Ford, Org. Sys. Coll. Vol. 3, 34(1955)). As another synthetic method, it can be synthesized from acrylonitrile and ammonia.

The salt of β-alanine is not limited, so long as it is a salt with an inorganic base or an organic base, or a salt with an acid, and is a salt acceptable for medicine, food, or cosmetic. Specific examples of the salt with the inorganic base or the organic base include a salt with an inorganic base, an organic base, or a metallic alkoxide. They can be prepared by mixing β-alanine with an inorganic base, an organic base, or a metallic alkoxide.

As the inorganic bases that can form salts, there may be mentioned a hydroxide, carbonate, hydrogen carbonate, acetate, or hydride of alkali metals (such as lithium, sodium, potassium, or the like); a hydroxide, hydride, or the like of alkaline earth metals (such as magnesium, calcium, or barium). As the organic bases that can form salts, there may be mentioned dimethylamine, triethylamine, piperazine, pyrrolidine, piperidine, 2-phenylethylamine, benzylamine, ethanolamine, diethanolamine, pyridine, collidine, or the like. Further, as the metallic alkoxide, there may be mentioned sodium methoxide, potassium tert-butoxide, magnesium methoxide, or the like. The salt of β-alanine is preferably a sodium salt, potassium salt, calcium salt, or a combination thereof.

Specific examples of the salt with an acid include a salt with an inorganic acid or an organic acid. As the inorganic acid that can form a salt, there may be mentioned hydrochloric acid.

<<Skin>>

The skin consists of three layers: epidermis, dermis, and subcutaneous tissue. The epidermis is mainly composed of cells called keratinocytes, and has a thickness of 0.06 to 0.2 mm.

The dermis is elastic and has blood vessels, nerves, and lymphatic vessels, and has a thickness of 2.0 to 2.2 mm. in addition, it contains cells such as mast cells and histiocytes. The dermis is mostly composed of a fibrous protein called collagen. The spaces therebetween are filled with a jelly-like matrix such as hyaluronic acid that holds water. In addition, a fibrous protein called elastin is contained to give elasticity to the skin. The cells that produce these fibers and substrates are called fibroblasts.

The subcutaneous tissue, which connects the skin to the lower tissues such as fascia, is a connective tissue with low fiber density.

The composition for inhibiting wrinkle of the present invention is not particularly limited, but preferably act on dermal fibroblasts, which are present in the dermis.

Specifically, type I collagen in the dermis can be increased and wrinkle formation in the skin can be inhibited, by acting on dermal fibroblasts, and then by promoting the generation of COL1A1, i.e. a type I collagen component, and/or inhibiting type I collagenase. In addition, elastin in the dermis can be increased and wrinkle formation in the skin can be suppressed, by acting on dermal fibroblasts, and then by promoting the synthesis of elastin and/or inhibiting elastase.

Furthermore, hyaluronic acid in the dermis can be increased and wrinkle formation in the skin can be suppressed, by acting on dermal fibroblasts, and then by promoting hyaluronic acid synthase and/or inhibiting hyaluronidase.

In the dermal cells, the above promotion of type I collagen component (COL1A1), inhibition of type I collagenase, promotion of elastin synthesis, promotion of microfibrillar associated protein (MFAP4), inhibition of elastase, promotion of hyaluronic acid synthase, or inhibition of hyaluronidase, can be examined by measuring an increase or decrease in mRNA (gene).

<<Muscle Cell-Mediated Inhibition>>

The composition for inhibiting wrinkle can act directly on dermal fibroblasts. On the other hand, the composition for inhibiting wrinkle of the present invention is not limited, but can act on dermal fibroblasts via a culture product (component) produced from muscle cells by the action of the composition for inhibiting wrinkle on muscle cells. When the wrinkle-inhibiting effect is exerted via muscle cells, myokines (IL-15, etc.) secreted by muscle cells may act on dermal fibroblasts, although it is not limited thereto.

In this specification, the “culture product” includes the culture supernatant and cultured cells obtained by contacting muscle cells with β-alanine or salt thereof. The muscle cells include cultured cells or muscle cells in the living body, but are preferably cultured cells. The cultured cell is not limited, but there may be mentioned a C2C12 cell, a primary cultured skeletal muscle cell, a HSkMC cell (skeletal muscle cell), a HSkMM cell (skeletal myoblasts), or an ioSkeletal Myocyte (skeletal muscle cell derived from human iPS cell).

The composition for inhibiting wrinkle of the present invention may comprise a culture product (substance) produced from muscle cells by β-alanine or salt thereof (hereinafter sometimes referred to as β-alanine or the like). The culture product (substance) produced from muscle cells by β-alanine or the like are not particularly limited as long as it has wrinkle-inhibiting effects, but, for example, includes an exosome. Therefore, the composition for inhibiting wrinkle of the present invention may comprise exosomes produced from muscle cells by β-alanine or the like.

The exosomes are a membrane-bound extracellular vesicle (EV) formed in the endosomal compartment of eukaryotic cells. A size of exosome is not limited, but are approximately 30 to 150 nm in diameter, and the majority is 100 nm or less. The exosome contains proteins, or RNA, and it is secreted out of the cell and may be taken up by other cells.

The formulation of the composition for inhibiting wrinkle of the present invention is not particularly limited. For example, oral agents, such as powders, subtle granules, granules, tablets, capsules, suspensions, emulsions, syrups, extracts, or balls; or parenteral agents, such as injections, liquid for external use, ointments, suppositories, creams for local administration, or eye-drops, there can be mentioned.

The above oral agent can be prepared in accordance with conventional methods, using excipients, such as gelatin, alginate sodium, starch, cornstarch, saccharose, lactose, glucose, mannitol, carboxymethyl-cellulose, dextrin, polyvinyl pyrrolidone, crystalline cellulose, soy lecithin, sucrose, fatty acid ester, talc, magnesium stearate, polyethylene glycol, magnesium silicate, silicic anhydride, or synthetic aluminum silicate; binders, disintegrators, surfactants, lubricants, flow accelerator, diluents, preservatives, colorants, flavors, corrigents, stabilizers, humectants, antiseptics, antioxidant, or the like.

Examples of the parenteral agents include injections. In a preparation of the injections, an aqueous solvent such as normal saline solution or Ringer solution, non-aqueous solutions such as plant oil or fatty acid ester, a tonicity agent such as glucose or sodium chloride, a solubility assisting agent, a stabilizing agent, an antiseptic agent, a suspending agent, or an emulsifying agent, can be optionally used, in addition to the active ingredient.

A dose of the composition for inhibiting wrinkle may be appropriately determined in accordance with, for example, age, sex, body weight, or degree of symptom of each patient, route of administration, or the like, and the determined dosage can be administered orally or parenterally. For example, in the case of an adult, the intake amount of the composition for inhibiting wrinkle of the present invention is preferably 0.01 to 100 mg/kg per day as β-alanine. In addition, as to the substance produced from muscle cells (e.g., exosomes) by β-alanine or the like, 0.01˜100 mg/kg/day is preferrable. The above administration method is an example, and other administration methods may be used. It is desirable that the administration method, dose, administration period, administration interval, and the like, of the composition for inhibiting wrinkle to humans are determined by a controlled clinical trial.

In addition, the dosage form is not limited to a drug medicine, but can be administered as a food composition (for example, functional food, health food, or beverage) or cosmetic composition as described below.

As a method for manufacturing the composition for inhibiting wrinkle containing β-alanine, known methods for manufacturing pharmaceutical composition, food composition, or cosmetic composition can be used, except that β-alanine is contained as an active ingredient. Further, known methods for manufacturing pharmaceutical composition, food composition, or cosmetic composition can be used, except that the culture product (substance; e.g., exosome) produced from muscle cells by β-alanine or the like is contained as an active ingredient.

The composition for inhibiting wrinkle of the present invention may contain other components. Examples of the other components include, for example, emulsifiers such as edible fats and oils, water, glycerin fatty acid ester, sucrose fatty acid ester, sorbitan fatty acid ester, propylene glycol fatty acid ester, glycerin organic acid fatty acid ester, polyglycerol fatty acid ester, calcium stearoyl lactylate, sodium stearoyl lactate, polyoxyethylene sorbitan fatty acid ester; thickening stabilizers such as locust bean gum, carrageenan, alginic acids, pectin, xanthan gum, crystalline cellulose, carboxymethyl cellulose, methyl cellulose, agar, glucomannan, gelatin, starch, or chemical starch; salty taste agents such as salt, or potassium chloride; acidulants such as acetic acid, lactic acid, or gluconic acid; sugars or sugar alcohols; sweeteners such as stevia or aspartame; colorants such as beta-carotene, caramel, or red koji pigment; antioxidants such as tocopherol or tea extract; food materials or food additives such as flavoring agent; pH adjuster; food preservative, or shelf life improver. Further, the composition for inhibiting wrinkle may contain various vitamins, or functional materials such as coenzyme Q, plant sterol, or milk fat globule membrane. The amount of these other components is preferably 80% by mass or less, more preferably 40% by mass or less, and further preferably 20% by mass or less, as a total amount in the composition for inhibiting wrinkle of the present invention.

<<Food Composition>>

The composition for inhibiting wrinkle of the present invention may be a food composition. The food compositions for inhibiting wrinkle of the present invention comprises β-alanine or a salt thereof. The food composition for wrinkle inhibition of the present invention may also contain culture product (substance; e.g., exosome) produced from muscle cells by β-alanine or the like. The food composition for inhibiting wrinkle of the present invention is not limited, as long as it can be administered orally.

The food in the food composition for inhibiting wrinkle of the present invention is a food or drink, and includes a beverage. The food in the present invention is not particularly limited, for example, there may be mentioned seasonings such as miso, soy sauce, sauce for noodles, sauce, soup stock, pasta sauce, dressing, mayonnaise, tomato ketchup, Worcestershire sauce, sauce for pork cutlet, or sprinkle; instant cooked foods such as a soup base, curry roux, white sauce, rice with tea base, or soup base; soups such as miso soup, soup, consomme soup, or potage soup; processed livestock products such as grilled meat, ham, or sausage; processed marine products such as boiled fish paste, dried fish, salted fish guts, fish boiled in soy sauce, rare delicacy; processed vegetable products such as pickles; snacks such as potato chips, or rice cracker; bakery foods such as bread, sweet bread, or cookies; cooked foods such as boiled foods, fried foods, grilled foods, curry, stew, gratin, rice, porridge, or rice ball; noodles such as pasta, wheat noodle, or ramen; fat processed foods such as margarine, shortening, fat spread, or flavored fat spread; materials for confectionery and bread such as flower pastes, or bean paste; mixed powders such as bread mix powder, cake mix powder, or fried food mix powder; confectioneries such as chocolate, candy, jelly, ice cream, or gum; Japanese confectioneries such as steamed bun, or castella; beverages such as coffee, coffee milk, tea, milk tea, soy milk, nutritional drink, vegetable drink, vinegared drink, juice, cola, mineral water, or sports drink; alcoholic beverages such as beer, wine, cocktail, or sour; milk and dairy products such as bovine milk, yogurt, or cheese.

The food composition for inhibiting wrinkle of the present invention can be prepared by using the known methods for manufacturing foods and drinks except for comprising β-alanine or the like, or except for comprising culture product (substance; e.g., exosome) produced from muscle cells by β-alanine or the like.

<<Cosmetic Composition>>

The composition for inhibiting wrinkle of the present invention may be a cosmetic composition. The cosmetic compositions for inhibiting wrinkle of the present invention comprises β-alanine or a salt thereof. It can inhibit wrinkle by comprising β-alanine and acting on dermal fibroblasts. In addition, the cosmetic compositions for inhibiting wrinkle of the present invention also contain culture product (substance; e.g., exosome) produced from muscle cells by β-alanine or the like. As the specifical cosmetics, there may be mentioned serums, cosmetic liquid, cleansing, emulsions, creams, lipsticks, foundations, gels, packs, white powders, blushes, hair tonics, shampoos, rinses, sunscreens, facial cleansers, or lip balms.

The amount of β-alanine or the like in the cosmetic composition of the present invention is not particularly limited as long as the effect of the present invention can be achieved, but is, for example, 0.1 to 100% by weight, preferably 1 to 50% by weight, and more preferably 1 to 25% by weight. The amount of culture product (substance; e.g., exosome) produced from muscle cells by β-alanine or the like in the cosmetic composition of the present invention is not particularly limited as long as the effect of the present invention can be achieved, but is, for example, 0.1 to 100% by weight, preferably 1 to 50% by weight, and more preferably 1 to 25% by weight.

The cosmetic composition of the present invention, as long as it does not inhibit the effects of the present invention, may contain moisturizing agents (e.g., trimethylglycine, N-[2-hydroxy-3-(trimethylammonio)propyl ]hydrolyzed wheat protein chloride, hyaluronic acid, sodium pyrrolidone carboxylic acid, betaine, jojoba oil, hydrolyzed keratin), colorants (e.g., pigment, or dye), viscosity modifiers (e.g., methylcellulose), emulsifying agents (e.g., glycerol monostearate), pearlescent agents (e.g., glycol distearate, or ethylene glycol distearate), salts (e.g., sodium chloride), plant extracts, preservatives (e.g. methylparaben, propylparaben, butylparaben, 1,3-butylene glycol (1,3-butanediol), phenoxyethanol, or pentylene glycol (1,2-pentanediol)), vitamins, fragrances, UV absorbers, antioxidants, wetting agents, chelating agent, pH adjuster (e.g. citric acid, or tartaric acid), and water.

<<Method for Inhibiting Wrinkle>>

The method for inhibiting wrinkle of the present invention comprises a step of administrating an effective amount of β-alanine or a salt thereof to a subject. The method for inhibiting wrinkle of the present invention may be performed as a medical practice. Preferably however, the method is not a medical practice. β-alanine or a salt thereof can be used in the method for inhibiting wrinkle. The formation of wrinkles can be inhibited by administering an effective amount of the composition for inhibiting wrinkle to humans or animals.

The method for inhibiting wrinkle of the present invention comprises a step of administrating an effective amount of a culture product secreted from muscle cells by β-alanine or salt thereof to a subject. The culture product is not limited, but includes an exosome. The method for inhibiting wrinkle of the present invention may be performed as a medical practice. Preferably however, the method is not a medical practice. The culture product (particularly, exosome) can be used in the method for inhibiting wrinkle. The formation of wrinkles can be inhibited by administering an effective amount of the composition for inhibiting wrinkle to humans or animals.

<<Culture Product>>

The culture product of the present invention can be produced by bringing β-alanine or salt thereof into contact with muscle cells. As the active ingredients in the culture product, there may be mentioned an exosome. However, there are also other active ingredients other than exosomes, and such active ingredients have not been identified at this time. Therefore, the present specification discloses culture products produced by bringing β-alanine or salt thereof into contact with muscle cells.

The exosome of the present invention can be produced by bringing β-alanine or salt thereof into contact with muscle cells. Therefore, the present specification discloses exosomes produced by bringing β-alanine or salt thereof into contact with muscle cells

<<β-Alanine Used in Method for Inhibiting Wrinkle>>

Said β-alanine can be used in the method for inhibiting wrinkle. That is, the present specification discloses β-alanine used in method for inhibiting wrinkle.

In addition, β-alanine can be used in promotion of type I collagen component (COL1A1), inhibition of type I collagenase, promotion of elastin synthesis, promotion of microfibrillar associated protein (MFAP4), inhibition of elastase, promotion of hyaluronic acid synthase, or inhibition of hyaluronidase.

<<Use of β-Alanine for Manufacturing Composition for Inhibiting Wrinkle>>

Said β-alanine or the like (culture product, in particular exosome) can be used for manufacturing the composition for inhibiting wrinkle. That is to say, the present specification discloses a use of β-alanine or the like for manufacturing the composition for inhibiting wrinkle.

In addition, β-alanine or the like can be used for manufacturing promotor of type I collagen component (COL1A1), inhibitor of type I collagenase, promotor of elastin synthesis, promotor of microfibrillar associated protein (MFAP4), inhibitor of elastase, promotor of hyaluronic acid synthase, or inhibitor of hyaluronidase.

<<Functions>>

In the composition for inhibiting wrinkle of the present invention, the mechanism by which wrinkle can be inhibited has not been fully elucidated, but may be presumed to be as follows.

In the dermis of the skin, there are many collagens, and jelly-like substrates such as hyaluronic acid fill the spaces therebetween. In addition, the presence of elastin, a fibrous protein, gives elasticity to the skin and suppresses the formation of wrinkles. As the skin ages, these collagen, hyaluronic acid, and elastin decrease, resulting in a decrease in skin elasticity and the formation of wrinkles. The composition for inhibiting wrinkle acts on dermal fibroblasts and can promote the production of collagen, hyaluronic acid, and elastin, and to suppress their degradation. Therefore, it is presumed that the formation of wrinkles in the skin can be inhibited.

In addition, it is considered that the composition for inhibiting wrinkle of the present invention containing β-alanine can act on muscle cells to produce substances (e.g., exosomes) effective for wrinkle inhibition from muscle cells. Therefore, it is presumed that the composition for inhibiting wrinkle can suppress the formation of wrinkles in the skin via the substances (e.g., exosomes) produced by muscle cells.

### EXAMPLES

The present invention will now be further illustrated by, but is by no means limited to, the following Examples.

### Example 1

In this Example, the effect of β-alanine on hyaluronic acid synthase (HAS2) in dermal fibroblasts was examined.

Skin fibroblasts (Skin Fibroblast (Immortalized Human Skin Fibroblast—SV40 (Applied biological materials, Richmond, BC, Canada)) were seeded at a concentration of 3.0×10⁴ cells/mL in 5 mL dish. After 24 hours of incubation, β-alanine (10 mM) was added thereto. After 24 hours, β-alanine was added secondary, and they were incubated for another 24 hours.

The medium was aspirated off and 1 mL/well of 1×PBS was added to wash the cells. Washing was performed twice. Then, 1×PBS (200 μL/well) and Lysis/-binding buffer (400 μL/well) were added to the entire dish, and the total volume was collected in a 1.5-mL tube. The collected samples were suspended using a vortex mixer for 60 seconds. The filter tube and the collection tube were assembled, the sample solution was added to the filter tube, and centrifuged at 4° C., 10000 rpm for 15 seconds. The liquid drained into the collection tube was discarded, and the filter tube and collection tube were assembled again. In a 1.5-mL tube, 90 μL of DNase Incubation Buffer per sample and 10 μL of DNase I per sample were mixed. The mixture was added to the filter tubes and incubated at room temperature for 15 minutes. After incubation, Wash buffer I (500 μL) was added to the filter tubes and centrifuged at 4° C., 10000 rpm for 15 seconds. The liquid drained into the collection tube was discarded, and the filter tube and the collection tube were reassembled. Wash buffer II (500 μL) was added to the filter tube and centrifuged at 4° C., 10000 rpm for 15 seconds. The liquid drained into the collection tube was discarded, and the filter tube and the collection tube were reassembled. Wash buffer II (200 μL) was added to the filter tube and centrifuged at 4° C., 13000×g for 2 minutes. The filter tube was inserted into a new 1.5-mL tube, and Elution buffer (70 μL) was added to the filter tube. The filter tube was left at room temperature for 3 minutes, and centrifuged at 4° C., 10000 rpm for 1 minute. The eluate obtained by the above procedure was used as the RNA solution. The concentration of RNA in the solution was calculated based on the absorbance value at 260 nm using a NanoDrop 2000/2000c spectrophotometer (Thermo Scientific, Waltham, MA, USA) and used in the subsequent experiments.

The cDNA was synthesized as follows. 0.5 μL of Oligo(dT)20 primer (10 pmoles/μL) was added to 1.0 μg of total RNA extracted from cells, and RNase-free water was added to the total volume to 13.5 L. The mixture was suspended by tapping, and spun down. Then, it was heat-treated with Thermal Cycler PTC-200 (MJ Research, Waltham, MA, USA) at 65 º C for 5 minutes, immediately transferred to ice and allowed to stand for 5 minutes. 4 μL of 5×RT Buffer (TOYOBO, Osaka, Japan), 2 μL of 10 mM dNTPs (GE Healthcare, Amersham Place, UK), and 0.5 μL of reverse transcription enzyme, ReverTra Ace (100 units/μL) (TOYOBO) were added to 0.2 mL tube per 1 sample, and mixed by pipetting. 6.5 μL of this mixture was added to the tube that was allowed to stand for 5 minutes, and the whole was mixed by pipetting on ice and then spun down. Then, a cDNA was synthesized by reacting at 42° C. for 20 minutes and 99° C. for 5 minutes, and used as a template for subsequent quantitative real-time PCR.

The quantitative RT-PCR was performed using the prepared cDNA as a template. 49 μL of sterile water, 3.5 μL of each of Primer Forward/Reverse (diluted to 10 μM), 7.0 μL of the template cDNA (diluted to 1/5 for HAS2, and diluted to 1/50 for β-actin) were added to a PCR tube (0.2 mL) on ice. The mixture was spun down, and then 24.5 μL of THUNDERBIRD SYBR qPCR Mix (TOYOBO) was added thereto and the whole was suspended well on ice. As a negative control, 17 μL of sterile water, 0.5 μL of each of Primer Forward/Reverse (diluted to10 μM) were added to a PCR tube (0.2 mL). The mixture was spun down, and then 7 μL of THUNDERBIRD SYBR qPCR Mix (TOYOBO) was added thereto and the whole was mixed well on ice. Thereafter, 25 μL of the mixture was added to each of 3 wells of a 96-well PCR Plate (NIPPON Genetics, Tokyo, Japan), and quantitative RT-PCR was performed using the Thermal Cycle Dicer Real Time System (Takara, Shiga, Japan). The PCR reaction was performed by 1 cycle of 95° C. for 30 seconds, and 40 cycles of 95ºC for 5 seconds and 60ºC for 60 seconds, and detected by FAM. The expression level of HAS2 was relatively quantified by comparing the Ct value of β-actin, which is one of the housekeeping genes, with the Ct value of HAS2 using the ΔΔCt method. The synthesis of the primers was outsourced to Sigma, and the sequences of the primers used were as follows.

HAS2 forward:
(SEQ ID NO: 1)
5′-GACAGGCATCTCACGAACCG-3′
HAS2 reverse:
(SEQ ID NO: 2)
5′-CCAACGGGTCTGCTGGTTTA-3′

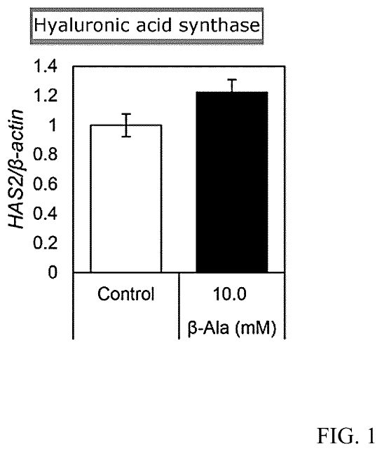

As shown in FIG. 1, the addition of β-alanine increased mRNA of hyaluronic acid synthase in dermal fibroblasts.

### Example 2

In this Example, muscle cells were treated with β-alanine, and the cell supernatant was added to dermal fibroblasts to examine their effects on collagen synthesis (type I collagen component; COL1A1) and degradation of collagen (type I collagenase; MMP-1).

C2C12 cells were seeded at a concentration of 2.0×10⁵ cells/mL in 5-mL dishes, and 48 hours later the medium was replaced with 2% HS-containing DMEM medium to induce differentiation. The culture medium was changed every 2 days and β-alanine (500 μM, 1 mM, or 10 mM) was added, and the culture supernatant was collected after 8 days of incubation. For controls, 1×PBS was added at a volume equal to that of β-alanine.

Dermal fibroblasts were seeded into 5-mL dishes at a concentration of 3.0×10⁴ cells/mL and cultured for 24 hours. The culture supernatant was removed and the collected supernatant of C2C12 cells was added at 5.0 mL/dish. At this time, 0.444 mL/dish of FBS was added to the culture medium to make up 10% of the culture medium together with HS contained in the supernatant of C2C12 cells. They were incubated for 48 hours.

Preparation of total RNA and synthesis of cDNA were performed in the same way as in Example 1. The primer sequences used were as follows.

COL1A1 forward:
(SEQ ID NO: 3)
5′-ACGAGACCAAGAACTGCCCC-3′
COL1A1 reverse:
(SEQ ID NO: 4)
5′-AGTGTCTCCCTTGGGTCCCT-3′
MMP-1 forward:
(SEQ ID NO: 5)
5′-CCCACAAACCCCAAAAGCGT-3′
MMP-1 reverse:
(SEQ ID NO: 6)
5′-GGGTAGAAGGGATTTGTGCGC-3′

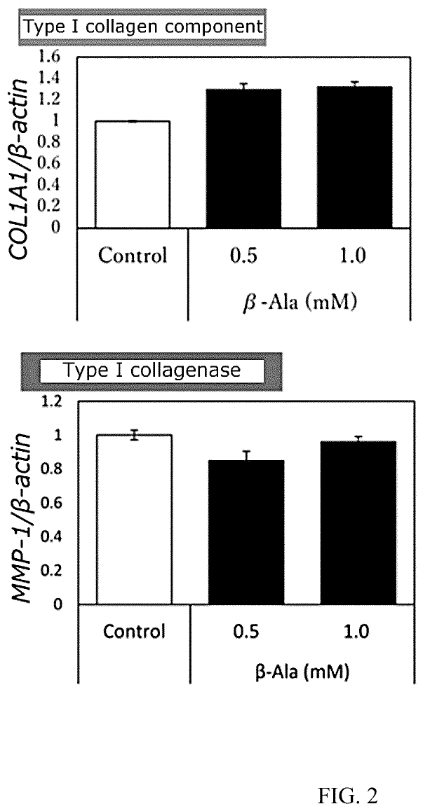

As shown in FIG. 2, the supernatant of C2C12 cells by β-alanine increased mRNA of COL1A1, and decreased mRNA of MMP-1 in dermal fibroblasts.

### Example 3

In this Example, muscle cells were treated with β-alanine, and the cell supernatant was added to dermal fibroblasts to examine their effects on elastin synthesis (ELN) and elastase (ELANE).

The procedure described in Example 2 was repeated except that the primers of ELN and ELANE were used instead of the primers of COL1A1 and MMP-1. The primer sequences used were as follows.

ELN forward:
(SEQ ID NO: 7)
5′-CGGTTCCAGGGGTTGTGTCA-3′
ELN reverse:
(SEQ ID NO: 8)
5′-GACACCAAAGCCGGGAAAGC-3′
ELANE forward:
(SEQ ID NO: 9)
5′-CACTGCGTGGCGAATGTAAAC-3′
ELANE reverse:
(SEQ ID NO: 10)
5′-CGTTGAGCAAGTTTACGGGGT-3′

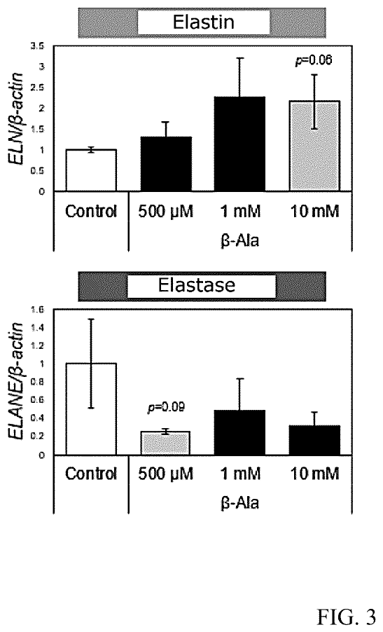

As shown in FIG. 3, the supernatant of C2C12 cells by β-alanine increased mRNA of ELN, and decreased mRNA of ELANE in dermal fibroblasts.

### Example 4

In this Example, muscle cells were treated with β-alanine, and the cell supernatant was added to dermal fibroblasts to examine their effects on hyaluronic acid synthase (HAS2) and hyaluronidase (HYAL1).

The procedure described in Example 2 was repeated except that the primers of HAS2 and HYAL1 were used instead of the primers of COL1A1 and MMP-1. The primer sequences used were as follows.

HAS2 forward:
(SEQ ID NO: 1)
5′-GACAGGCATCTCACGAACCG-3′
HAS2 reverse:
(SEQ ID NO: 2)
5′-CCAACGGGTCTGCTGGTTTA-3′
HYAL1 forward:
(SEQ ID NO: 11)
5′-GAATGCCAGCCTGATTGCCC-3′
HYAL1 reverse:
(SEQ ID NO: 12)
5′-TGTCCCAGTTGAAGGCCCAG-3′

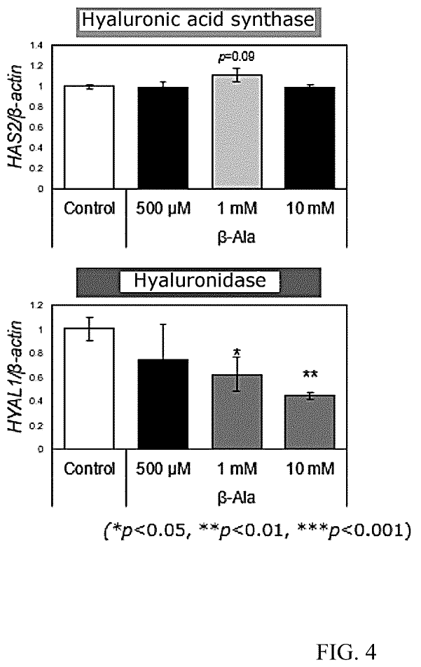

As shown in FIG. 4, the supernatant of C2C12 cells by β-alanine increased mRNA of HAS2, and decreased mRNA of HYAL1 in dermal fibroblasts.

### Example 5

In this example, muscle cells were treated with β-alanine, and exosomes were collected from the cell supernatant. The collected exosomes were added to dermal fibroblasts to examine their effects on collagen synthesis (type I collagen component; COL1A1), hyaluronic acid synthase (HAS2), elastin synthesis (ELN), and microfibrillar associated protein (MFAP4).

C2C12 cells were seeded in 10 mL dishes at 2×10⁵ cells/mL. The cells were cultured in DMEM medium containing 10% Exosome-depleted FBS Media Supplement Heat Inactivated. After 48 hours, the medium was replaced with DMEM medium containing 2% serum Exosome-depleted FBS Media Supplement Heat Inactivated to induce differentiation. After another 24 hours, the medium was replaced with DMEM medium containing 2% Exosome-depleted FBS Media Supplement Heat Inactivated. The medium was changed every 2 days thereafter, and the medium was changed and β-alanine was added on days 8 and 9 after cell seeding. As the β-alanine, β-alanine prepared at 100 mM in DMEM medium containing 2% Exosome-depleted FBS Media Supplement Heat Inactivated was used, and the medium was removed by the amount of β-alanine added. Exosomes were purified from the supernatant at 10 days after seeding. The control supernatant was the untreated supernatant in which β-alanine was not added but only the medium was exchanged.

For each sample, the supernatant of two 10-mL Dish sheets was collected. Cells in the culture supernatant were removed by centrifuging 10 mL of the collected culture supernatant at 300×g for 5 minutes. The supernatant was transferred to another tube, and then centrifuged at 1,200×g for 20 minutes to remove cellular fragments. The supernatant was transferred to another tube and centrifuged at 10,000×g for 30 minutes to remove extracellular vesicles larger than exosomes. The culture supernatant from which cells and large extracellular vesicles were removed was concentrated approximately 40-fold by a centrifugal ultrafiltration unit (AmiconUltra-15 100K, Merck Millipore) using a filter with a molecular weight of 100,000. MagCapture Exosome Isolation PS Kit Ver.2 (FUJIFILM Wako) was used for purification of exosomes from the concentrated culture supernatant.

First, a buffer was prepared. 0.55 mL of Exosome Immobilizing/Washing Buffer (10×) and 4.95 mL of purified water were added to a 15-mL tube, and then Exosome Immobilizing/Washing buffer (1×) was prepared by adding 11 μL of Exosome Binding Enhancer (500×). Exosome Elution Buffer (1×) was prepared by adding 15 μL of Exosome Elution Buffer (10×) and 135 μL of purified water to a 1.5-mL tube. To the Exosome Elution Buffer, EV-Save TM Extracellular Vesocle Biocking Reagent (FUJIFILM Wako) was added as an exosome protection agent at a dilution of 1/100.

Next, Exosome Capture immobilized beads were prepared. Sixty microliters of Biotin Capture Magnetic Beads which is agitated well by a vortex mixer, were placed in a 1.5-mL reaction tube, and 500 μL of Exosome Immobilizing/Washing buffer (1×) was added to the tube, and the mixture was suspended by a vortex mixer. Then, the tube was spun down and set on a magnetic stand, and allowed to stand for about 1 minute. Next, 500 μL of Exosome Immobilizing/Washing buffer (1×) and 10 μL of Biotin-labeled Exosome Capture were added to the tube, and the tube was removed from the magnetic stand, and suspended by a vortex mixer. Then, the tube was reacted for 10 minutes by inverting and mixing by a rotating stirrer at room temperature. The tube was spun down, set on the magnetic stand again, and allowed to stand for about 1 minute. When the magnetic beads completely adhered to the tube wall, the supernatant was removed with a pipette. (a) 500 μL of Exosome Immobilizing/Washing buffer (1×) was added to the tube, and the tube was removed from the magnetic stand, suspended by a vortex mixer, spun down, set on the magnetic stand again, and allowed to stand for 1 minute. When the magnetic beads completely adhered to the tube wall, the supernatant was removed with a pipette. The above procedure (a) was repeated one more time. The Exosome Capture immobilized beads were completed by the above procedure.

Next, the Exosome Capture immobilized beads were reacted with the culture supernatant concentrated as described above. About 500 μL of the 40-fold concentrated culture supernatant was transferred to a sterile 1.5-mL tube, and 1/500 volume of Exosome Binding Enhancer (500×) was added to the cell supernatant and mixed using a vortex mixer. The tube was spun down and the sample was transferred to a tube containing Exosome Capture immobilized beads (Reaction Tube) and mixed by a vortex mixer. The reaction was carried out at room temperature for at least 1 hour while mixing invertedly with a rotary stirrer. After spinning down the 1.5-mL Reaction tube, it was set on the magnetic stand and allowed to stand for about 1 minute. When the magnetic beads completely adhered to the tube wall, the supernatant was removed with a pipette and the exosomes were bound to the beads.

Next, the exosome-binding beads were washed. (b) Add 1 mL of Washing Buffer containing Exosome Binding Enhancer to a 1.5 mL Reaction tube containing exosome-binding beads, and the mixture was suspended by a vortex mixer. The 1.5 mL Reaction tube was spun down and set on a magnetic stand, and allowed to stand for about 1 minute.

When the magnetic beads completely adhered to the tube wall, the supernatant was removed.

Further, the above procedure (b) was repeated twice. The washed exosome-bound beads were prepared by this procedure.

Exosomes were eluted by the following procedure. 50 μL of Exosome Elution Buffer (1×) was added to a 1.5 mL Reaction tube containing washed exosome-bound beads, and then the Reaction tube was removed from the magnetic stand, and suspended by a vortex mixer. The tube was spun down, set on the magnetic stand, and allowed to stand for 1 minute. After the magnetic beads had completely adhered to the tube wall, the supernatant was collected into a new sterile 1.5-mL tube. Further, 50 μL of Exosome Elution Buffer (1×) was added to the magnetic beads remaining in the 1.5-mL reaction tube, and the tube was removed from the magnetic stand, and suspend by a vortex mixer. The tube was spun down, set on the magnetic stand, and allowed to stand for 1 minute. When the magnetic beads completely adhered to the tube wall, the supernatant was collected in a new sterile 1.5-mL tube (as described above), to be a total of 100 μL of exosome solution.

Dermal Fibroblasts were seeded at a concentration of 3.0×10⁴ cells/mL in 5-mL dishes and cultured for 24 hours. The culture supernatant was removed, and purified exosomes were added at 1 μg/well and cultured for 48 hours.

Preparation of total RNA and synthesis of cDNA were performed in the same way as in Example 1. The primer sequences of microfibrillar associated protein (MFAP4) were as follows.

MFAP4 forward:
(SEQ ID NO: 13)
5′-GAATGCCAGCCTGATTGCCC-3′
MFAP4 reverse:
(SEQ ID NO: 14)
5′-TGTCCCAGTTGAAGGCCCAG-3′

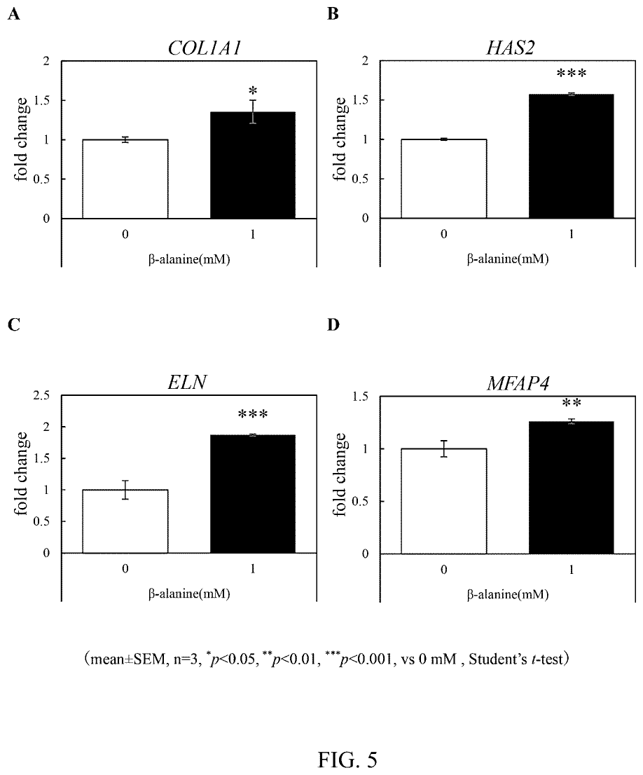

As shown in FIG. 5, the exosomes from differentiated C2C12 cells treated with 1 mM β-alanine increased expressions of type I collagen component (COL1A1), hyaluronic acid synthase (HAS), elastin (ELN), and microfibrillar associated protein (MFAP4), which are genes that work to inhibit wrinkles.

### Example 6

In this example, muscle cells were treated with β-alanine, and exosomes were collected from the cell supernatant. The collected exosomes were added to dermal fibroblasts to examine their effects on collagenase (MMP-1), hyaluronidase (HYAL1), and elastase (ELANE).

The procedure described in Example 5 was repeated except that the primers of MMP-1, HYAL1, and ELANE were used instead of the primers of COL1A1, HAS2, ELN, and MFAP4.

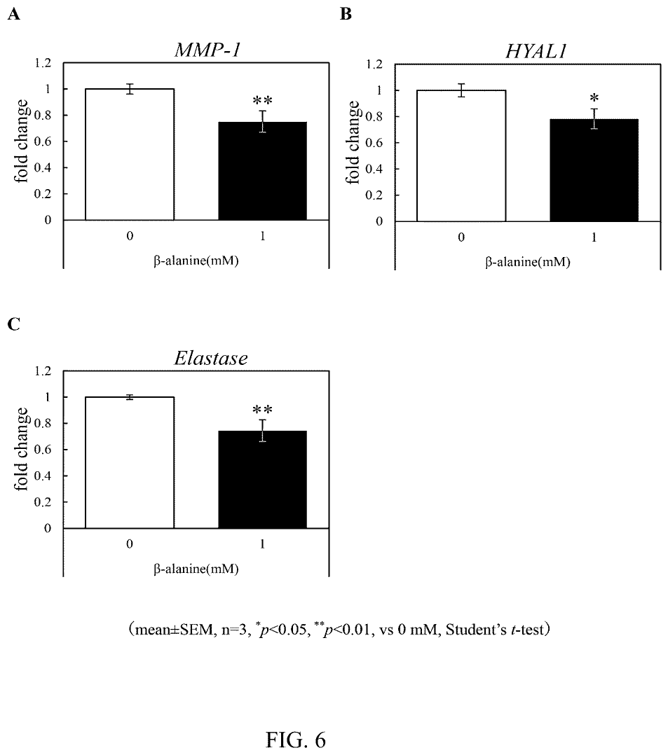

As shown in FIG. 6, the exosomes from differentiated C2C12 cells treated with 1 mM β-alanine significantly decreased expressions of collagenase (MMP-1), hyaluronidase (HYAL1), and elastase (ELANE), which are genes that work to promote wrinkles.

### Example 7

In this example, at the protein level, the enhanced expression of COL1A1 in dermal fibroblasts treated with exosomes was examined by immunostaining.

Dermal Fibroblasts were seeded onto 96-well Black plates (Greiner Bio-one, Tokyo, Japan) at 6.0×10⁴ cells/well and cultured in DMEM medium containing 10% FBS. After 24 hours, 1 μg/well of exosomes purified as described in Example 5 was added. As a control, exosomes purified from the culture supernatant of differentiated C2C12 cells that were induced to differentiate without β-alanine were used. After 48 hours, 8% paraformaldehyde was added at 100 μL/well, so as to be at a final concentration of 4%, and the cells were fixed by incubation at room temperature for 15 minutes. After 15 minutes, the fixative solution was removed and the cells were washed three times with 1×PBS for 5 minutes.

Then, blocking buffer was added thereto at 200 μL/well and the wells were blocked at 37° C. for 1 hour. After the buffer was removed, primary antibody (COL1A1 (E8F4L) XP (registered trademark) Rabbit mAb, #72026: 200× dilution) diluted with antibody dilution buffer was added at 100 μL/well and incubated at 4° C. overnight. Then, the cells were washed three times with 100 μL/well of 1×PBS for 5 minutes. The following procedures were performed under light-shielded condition. Secondary antibody (Anti-rabbit IgG (H+L),F(ab′)2 Fragment (Alexa Fluor (registered trademark) 488 Conjugate), #4412) diluted 1,000-fold with antibody dilution buffer was added to cells at 100 μL/well. The cells were incubated at room temperature for 2 hours. After washing the cells three times with PBS for 5 minutes, a 500-fold dilution of Cellstain (registered trademark)-Hoechst 33342 solution (Dojindo, Kumamoto, Japan) was added at 100 μL/well, and the cells were allowed to stand at room temperature for 30 minutes for nuclear staining. After 30 minutes, Cellstain (registered trademark)-Hoechst 33342 solution was removed, washed twice with 1×PBS, and 100 μL of PBS was added to each well. Thereafter, images were acquired by IN Cell Analyzer 2200 (GE Healthcare Japan, Tokyo, Japan).

Images were analyzed by IN Cell Investigator High-content image analysis software (GE Healthcare Japan) using the protocol “Mito tracker_Red_Green_C2C12_210210 20×”.

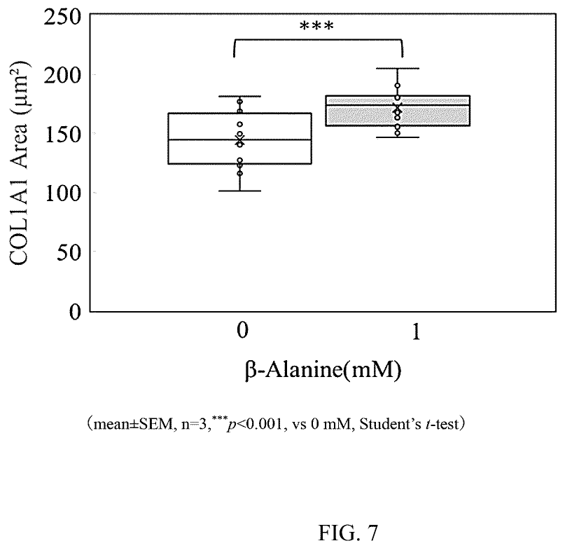

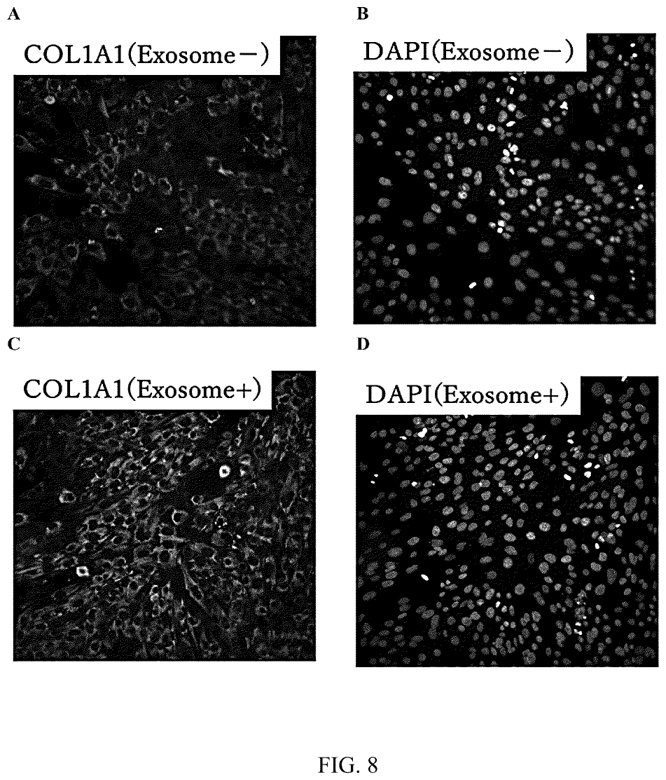

As shown in FIG. 7, the addition of exosomes significantly increased the area of type I collagen components. FIG. 8 shows a photograph taken by IN Cell Analyzer 2200 at the time of analysis.

### INDUSTRIAL APPLICABILITY

The composition for inhibiting wrinkle of the present invention inhibits wrinkle formation in the skin and can be used as a pharmaceutical composition, food composition, or cosmetic composition.

1. A composition for inhibiting wrinkle, comprising β-alanine or a salt thereof as an active ingredient.

2. The composition for inhibiting wrinkle according to claim 1, wherein an inhibition of wrinkle is due to, in dermal cells, a promotion of type I collagen component (COL1A1), inhibition of type I collagenase, promotion of elastin synthesis, promotion of microfibrillar associated protein (MFAP4), inhibition of elastase, promotion of hyaluronic acid synthase, or inhibition of hyaluronidase.

3. A composition for inhibiting wrinkle, comprising a culture product secreted from muscle cells by β-alanine or salt thereof.

4. The composition for inhibiting wrinkle according to claim 3, wherein an inhibition of wrinkle is due to, in dermal cells, a promotion of type I collagen component (COL1A1), inhibition of type I collagenase, promotion of elastin synthesis, promotion of microfibrillar associated protein (MFAP4), inhibition of elastase, promotion of hyaluronic acid synthase, or inhibition of hyaluronidase.

5. The composition for inhibiting wrinkle according to claim 3, wherein the culture product is an exosome.

6. The composition for inhibiting wrinkle according to claim 1, wherein the composition is a food composition or a cosmetic composition.

7. A method for inhibiting wrinkle, comprising a step of administrating an effective amount of β-alanine or a salt thereof to a subject.

8. A method for inhibiting wrinkle, comprising a step of administrating an effective amount of a culture product secreted from muscle cells by β-alanine or salt thereof to a subject.

9. A method for inhibiting wrinkle according to claim 8, wherein the culture product is an exosome.

10. The composition for inhibiting wrinkle according to claim 2, wherein the composition is a food composition or a cosmetic composition.

11. The composition for inhibiting wrinkle according to claim 3, wherein the composition is a food composition or a cosmetic composition.

12. The composition for inhibiting wrinkle according to claim 4, wherein the composition is a food composition or a cosmetic composition.

13. The composition for inhibiting wrinkle according to claim 5, wherein the composition is a food composition or a cosmetic composition.
